(19) United States
(12) Patent Application Publication
(10) Pub. No.: US 2018/0177570 A1
(43) Pub. Date: Jun. 28, 2018

(54) LIGHT ASSISTED ORTHODONTIC DEVICES AND METHODS OF MAKING AND USING SAME

(71) Applicant: Ormco Corporation, Orange, CA (US)

(72) Inventors: Sammel Shahrier Alauddin, Rancho Cucamonga, CA (US); Jessica Elivier Grande, Chino, CA (US); Jie Chen, Indianapolis, IN (US); Ian Kitching, Rancho Cucamonga, CA (US)

(21) Appl. No.: 15/855,629

(22) Filed: Dec. 27, 2017

Related U.S. Application Data
(60) Provisional application No. 62/439,688, filed on Dec. 28, 2016.

(51) Int. Cl. A61C 7/08; A61N 5/06; A61C 19/06

(52) U.S. Cl. CPC A61C 7/08; A61N 5/0603; A61C 19/06; A61N 2005/067; A61N 2005/0659; A61N 2005/0626; A61N 2005/0653; A61N 2005/0606

(57) ABSTRACT

A light assisted orthodontic device includes at least one material layer formed to be positioned over one or more teeth; a light source coupled to the at least one material layer, the light source being configured to emit light having a wavelength of 700 nm to 1500 nm; and a power source configured to power the light source. A method for making a light assisted orthodontic device includes forming a first material layer shaped to be positioned over one or more teeth; and coupling a light therapy array to the first material layer. A method of orthodontic treatment includes positioning the device over one or more teeth, the device applying a corrective pressure to the one or more teeth; and emitting light having a wavelength of 700 nm to 1500 nm to the one or more teeth.

### CROSS REFERENCE TO RELATED APPLICATION

This application claims priority to U.S. Provisional Patent Application Ser. No. 62/439,688 filed Dec. 28, 2016, the disclosure of which is incorporated by reference herein in its entirety.

### TECHNICAL FIELD

The invention relates generally to orthodontic devices, and more particularly to light assisted orthodontic devices.

### BACKGROUND

Orthodontic appliances, and in particular orthodontic brackets, represent a principal component of corrective orthodontic treatments devoted to improving a patient's malocclusions, crookedness, and other flaws of the teeth. In conventional orthodontic treatment, an archwire interacts with orthodontic brackets and applies corrective forces that coerce the teeth to move into orthodontically correct positions. Conventional orthodontic brackets are ordinarily formed from stainless steel, which is strong, nonabsorbent, weldable, and relatively easy to form and machine. Patients undergoing orthodontic treatment using metal orthodontic brackets, however, may be embarrassed by the visibility of metal, which is not cosmetically pleasing.

Another method of orthodontic treatment may use a series of clear, removable teeth aligners as an alternative to orthodontic braces. The series of aligners are successively worn by the patient to reposition the teeth to correct flaws. During a treatment process, a dental practitioner may prescribe a series of aligners, which are generally placed over but are not themselves adhesively secured or otherwise attached to the patient's teeth, to move one or more teeth from their original position to an aesthetically pleasing position. A series of aligners may be used to treat the patient because the degree of movement produced by an aligner is limited. Each aligner in a series may be designed to move one or more teeth over a portion of the entire distance towards the desired position. An aligner for orthodontic treatment is also referred to as an orthodontic aligner and it is a type of incremental position adjustment dental appliance.

The addition of low level light technology to traditional or modern orthodontic treatment provides a biologically advanced pathway for tooth movement. Forces (and their corresponding moments) from orthodontic appliances drive the bone remodeling process at the tooth root, facilitating the established orthodontic treatment process. Low level light therapy (photobiomodulation) for orthodontics is mainly driven by infrared wavelengths applied to mandibular and maxillary bone tissue to stimulate advanced cellular activity. In that regard, light energy can stimulate osteoblast and osteoclast differentiation and proliferation, which drive bone remodeling. The increased bone remodeling accelerates tooth movement if the tooth is under an orthodontic force or strengthen the bone if no orthodontic treatment is involved. It is also believed that light energy reduces pain response in biological tissues, a common side effect of orthodontics. Accelerated orthodontics is among the most desired clinical needs to both patients and doctors.

Consequently, there is a need for improved orthodontic devices with osseo-stimulative luminescence.

### SUMMARY

The present invention overcomes the foregoing and other shortcomings and drawbacks of orthodontic treatment. While the invention will be described in connection with certain embodiments, it will be understood that the invention is not limited to these embodiments. On the contrary, the invention includes all alternatives, modifications and equivalents as may be included within the scope of the present invention.

In an embodiment, a light assisted orthodontic device includes at least one material layer formed to be positioned over one or more teeth; a light source coupled to the at least one material layer, the light source being configured to emit light having a wavelength of 700 nm to 1500 nm; and a power source configured to power the light source.

In an embodiment, a method for making a light assisted orthodontic device includes forming a first material layer shaped to be positioned over one or more teeth; and coupling a light therapy array to the first material layer, the light therapy array comprising a light source configured to emit light having a wavelength of 700 nm to 1500 nm.

In an embodiment, a method of orthodontic treatment using a light assisted orthodontic device includes positioning the device over one or more teeth, the device applying a corrective pressure to the one or more teeth; and emitting light having a wavelength of 700 nm to 1500 nm to the one or more teeth.

In an embodiment, a method for determining a penetration rate of light through oral tissue includes providing a light source; directing light from the light source onto a sample of oral tissue; measuring a first power of the light at a first surface of the sample; and measuring a second power of the light at a second surface of the sample. The method further includes computing a surface loss of the light; and determining the penetration rate of the light using the first power, the second power, and the surface loss.

### BRIEF DESCRIPTION OF THE DRAWINGS

The accompanying drawings, which are incorporated in and constitute a part of this specification, illustrate embodiments of the invention and together with the detailed description given below, serve to explain various aspects of the invention.

FIG. 1 is a perspective view of a light assisted orthodontic device, shown as an aligner, in accordance with an embodiment of the invention.

FIG. 2A is a schematic diagram of the formation of an inner layer of the aligner of FIG. 1 on a positive mold according to one embodiment of the invention.

FIG. 2B is a side view of features being attached to the inner layer of FIG. 2A.

FIG. 2C is a schematic diagram of the formation of an outer layer over the inner layer and features of FIG. 2B.

FIG. 3 is a cross-sectional view of the multi-layer aligner of FIG. 2C taken along section line 3-3.

FIG. 4 is a perspective view of the multi-layer aligner of FIG. 2C shown removed from the positive mold.

FIG. 5 is a perspective view of a 3-D digital model of a patient's arch and fixed orthodontic appliances.

FIG. 6A is a perspective view of a light assisted orthodontic device, shown as a tray, in accordance with an embodiment of the invention.

FIG. 6B is a perspective view of the light assisted orthodontic device of FIG. 6A shown on a positive mold based on the 3-D digital model of FIG. 5.

FIGS. 7 and 8 are schematic diagrams of a system for testing the penetration rate of the oral tissue complex.

FIG. 9 is a graph showing surface loss rate of the gum.

FIG. 10 is a graph showing attenuation coefficients of the gum, cortical bone, and trabecular bone.

FIG. 11 is a graph showing error of penetration compared to measurement for tissue combinations of: 1) gum and cortical bone and 2) cortical bone and trabecular bone.

FIG. 12 is a graph showing error of penetration to measurement of natural anatomy on a pig.

### DETAILED DESCRIPTION

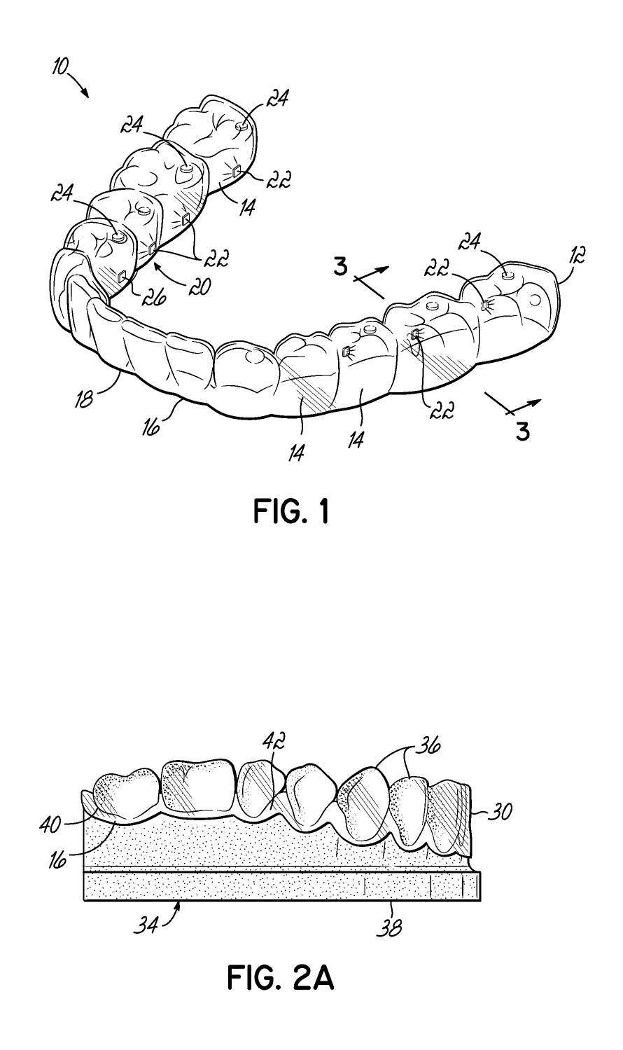

Referring now to the drawings, and to FIGS. 1-4 in particular, in one embodiment, the light assisted orthodontic device is an aligner 10 capable of moving teeth. In particular, the aligner 10 may move one or more teeth from one orientation to another. Overall, the aligner 10 moves the teeth toward an orientation whereby the teeth ultimately are positioned at their final orthodontically correct and aesthetic positions. Tooth movement may be according to a predetermined treatment plan. As shown in FIG. 1, the aligner 10 includes a hollow shell 12 that is configured to encapsulate one or more crowns of a patient's teeth. The shell 12 is formed with a plurality of cavities 14 that collectively define an edge 16. The edge 16 in turn defines an opening 18 in the shell 12. Each cavity 14 is shaped to receive a specific one of the patient's teeth through the opening 18 such that the edge 16 is positioned proximate the patient's gingiva. The aligner 10 applies a corrective pressure to one or more teeth.

With reference to FIG. 1, in one embodiment, the aligner 10 includes at least a light source 20 capable of emitting infrared light. The light source 20 may be configured to light a region that may be, but is not limited to, one or more teeth (i.e., incisor, canine, premolar, molar, etc.), and that may include the root of one or more teeth, the gum, the alveolar bone, the cortical bone, the trabecular bone, etc. The dose of the light emitted by the light source 20 may, for example, range from about 1 J/cm² to less than 5 J/cm², from about 1 J/cm² to about 2 J/cm², and may be about 1 J/cm². The dose stimulates the proliferation of osteoblast, osteoblast β-catenin signaling response for bone formation, and the osteoclast's differentiation and bone resorption activity. Further, the dose does not negatively affect the viability of osteoblast or osteocyte cells, while a dose of 5 J/cm² does cause cell death, which may attribute to reduced bone resorption activity. The wavelength emitted by the light source 20 may range from, for example, 700 nm to 1500 nm, 700 nm to 1050 nm, or 1050 nm to 1500 nm. These wavelengths of light penetrating osteocytes are believed to accelerate ATP (Adenosine Tri Phosphate) production in cellular mitochondria. This additional energy accelerates the bone remodeling process and reduces overall treatment time. Of note, the penetration rate of the emitted light may vary based on the target tissue, as described further in the Example. Penetration rates of a wide spectrum of light may be characterized by the attenuation coefficient regardless of thickness for three oral tissues—the gum, cortical bone, and trabecular bone. For these oral tissues, the penetration rate is inversely proportional to the attenuation coefficient. Trabecular bone, cortical bone, and gum (in that order) have the highest penetration rates. Thus, the penetration rates are tissue type dependent. For these oral tissues, longer wavelengths, especially greater than 700 nm, demonstrated higher penetration rates. The light intensity may range from, for example, 0.3 mW/cm² to 8.8 mW/cm². In an embodiment of the present invention, the specific parameters of the light emitted by the light source 20 to penetrate the oral tissue complex and stimulate targeted cellular activity in osteocytes (bone) supporting the tooth root may be determined according to a derived prediction equation, as described further in the Example below.

With further reference to FIG. 1, in the illustrated embodiment, the light source 20 includes an array of diodes 22 that are configured to emit infrared light toward the lingual surface of the oral cavity, primarily the alveolar bone surrounding the dental roots. In an embodiment, the wavelength or dosage of each of the diodes 22 may vary from diode to diode to increase device performance. For example, diodes in the posterior regions of the aligner 10 may be designed to emit greater wavelengths to overcome thicker oral tissue complex compared to anterior regions of the aligner 10 (anterior being in the labial direction of posterior). Alternatively, these diodes may require more frequent operation to optimize light dosage received by the target tissues. The diodes 22 may be, for example, light emitting diodes (LEDs), surface mounted diodes (SMDs), or Schottky diodes. The diodes 22 may be connected in series or in parallel. The diodes 22 may be any size ranging from, for example, 200 μm by 200 μm by 75 μm to 5 mm by 5 mm by 200 μm. For example, the size of the diodes 22 may be 0402, 0606, 0805, 1005, 1206, 1210, 1806, or 1812.

It should be recognized that the light source 20 may vary. In an embodiment, the light source 20 may include one or more organic light emitting diodes (“OLEDs”) in which the emissive electroluminescent layer is a film of organic compound that emits light in response to an electric current. This technology is commonly used in advanced electronics including flat screen televisions and mobile phones. An OLED may be based on small molecules and/or those employing polymers. The OLED may be transparent. In another embodiment, the light source 20 may include one or more polymer-light emitting electrochemical cells (LECs). The LECs may be designed in numerous shapes to fit in the aligner 10. The LECs may additionally use quantum dots to select specific wavelengths to be applied. In another embodiment, the light source 20 may include one or more laser diodes. The laser diodes may scatter light through a diffuser (e.g., on the lingual side of the teeth) that will multiply the light towards the desired tissue. In another embodiment, the light source 20 may include an optoelectronic device that can source, detect, and control light. In another embodiment, the light source 20 may be made of polymer LEDs. Polymer LEDs may stay lit up when stretched and scrunched for flexibility in the mouth. The polymer substrate can be as thin as a few hundred nanometers.

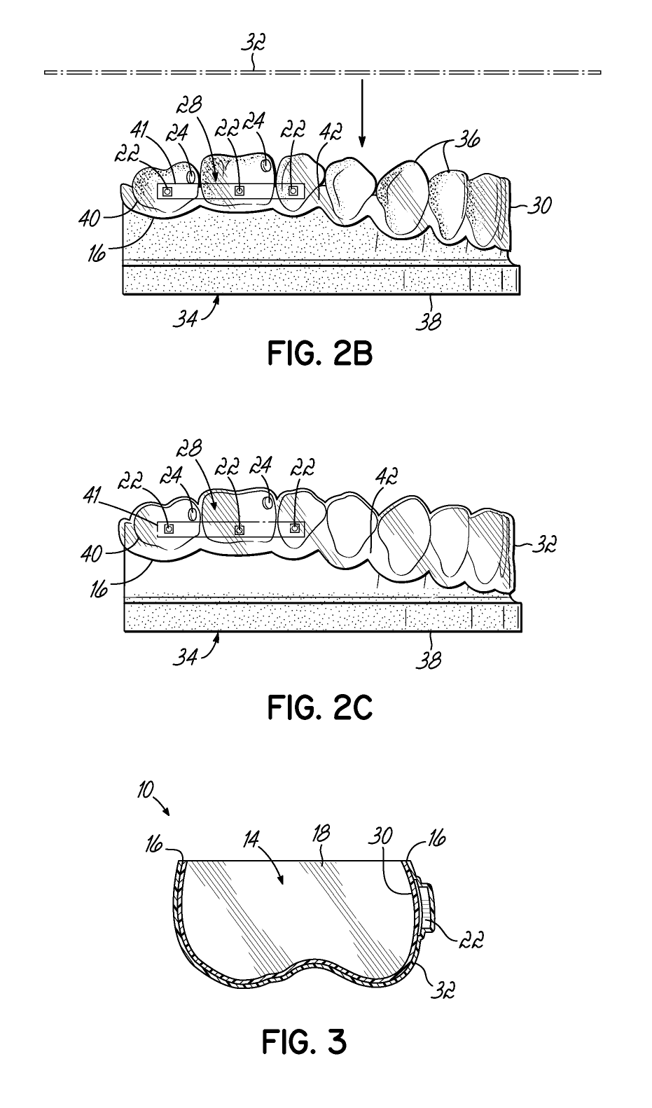

In an aspect of the present invention, the light source 20 may be carried via a substrate. For example, in an embodiment, the light source 20 may be printed on a flat or flexible substrate via thin-film processing. The substrate may be made of, for example, glass, plastic, or rubber and may be transparent. For example, the diodes 22 may be connected to each other via a flexible strip (e.g., as shown in FIG. 2B). Further, resistors and chips may be embedded with the diodes 22 in the flexible strip. The flexible strip can have a width of, for example, 0.5 mm to 5 mm, an arch length of up to 100 mm, and a thickness of no greater than 1 mm. The number of diodes 22 on the flexible strip may range from 1 to 2,500. The number of diodes 22 depends at least in part on the intensity that is desired. In an embodiment, an ultrathin, flexible optoelectronic device may include diodes 22 the size of individual neurons and can be placed in a stretchable sheet. In an embodiment, the diodes 22 may be soldered together.

With further reference to FIG. 1, the aligner 10 includes a power source 24 configured to operate in an environment with a temperature of about 37° C. (e.g., in a patient's mouth). In an embodiment, the power source 24 may be a standard lithium ion battery. It should be recognized that, while FIG. 1 shows the power source 24 being embedded in the aligner 10, the power source 24 may be separate from the aligner 10.

In another embodiment, the power source 24 may be a high energy density rechargeable battery that may be made of, but is not limited to, ZincPoly, Ni—Cd, Ni-MH, and Li—Si Nanowire. The high energy density rechargeable battery may be ultrathin, flexible, and/or customizable. For example, in an embodiment, the battery can be made of any moldable material, such as a composite structure of two conductive or semi-conductive sheets sandwiching a thin polymer insulator all bonded at the interfaces. The moldable material may have a protective coating over the outer conductive sheets to improve durability of the composite structure. Further, the moldable material may be patterned into custom shapes.

In another embodiment, the power source 24 may be a micro battery that can be made of, but is not limited to, lithium/carbon fluoride. Other possible cathodes include MnO₂, I₂, FeS₂, or SOCl₂, for example. Micro batteries can be encapsulated in various forms including, without limitation, a cylindrical capsule and a flexible strip. Further, a micro battery can be as thin as a human hair or as thick as a grain of rice.

In another embodiment, the power source 24 may be a biodegradable battery that dissolves in the body. The biodegradable battery can be made of, but is not limited to, magnesium, iron, tungsten, or molybdenum. The time taken for the biodegradable battery to dissolve can be controlled by changing the size of the battery, the temperature-degradation profile, and adding a coating. The biodegradable battery can be implanted and does not have to be embedded within the aligner 10.

In another embodiment, the power source 24 may be a microbial fuel cell that will generate power upon contact with, for example, saliva or glucose. These fuel cells contain anaerobic bacteria at the anode that consume organic matter (e.g., saliva) in the process of generating electrons. The microbial fuel cell can be any shape or size and will not contain organisms that are toxic to the human body. A microbial fuel cell may need contact with the saliva to generate power.

In another embodiment, the power source 24 may be a nanowire super-capacitor electrode that can be made of, but is not limited to, copper. The nanowire will not only power the light source 20 but may also provide support at the inside of the aligner 10. The nanowire can be made in various shapes including, but not limited to, rectangular or cylindrical.

In another embodiment, the power source 24 may be made of a piezoelectric material that generates electricity when a certain stress is applied (i.e., bending, tensile, compression). In other words, the power source 24 may harvest the power from stress applied to the power source 24 during use of the aligner 10. This piezoelectric material can be, for example, a ceramic (e.g., barium titanate) or a polymer (e.g., polyvinylidene fluoride). The piezoelectric material can be in any shape or size and may be flexible or rigid. The piezoelectric material may be flexible, stretchable, and highly sensitive. The piezoelectric material may be ultra-sensitive, such that it creates electricity upon simple motion of the jaw when the user talks or moves. The location of the piezoelectric material may vary based on its configuration. For example, the piezoelectric material may be positioned between the molars at the occlusal surface (contact). If the piezoelectric material is a polymer and is thin enough (e.g., less than 0.5 mm), it can be positioned on the gums. If the polymer is flexible enough, it can be embedded within the aligner 10.

In an aspect of the present invention, a light assisted orthodontic device may include a rechargeable power source. When the device is designed to be an aligner (e.g., aligner 10), the power source may be recharged when the patient removes the aligner, such as during eating or teeth brushing. If the device is designed to be worn at night, the power source may be charged during the day when the device is not being worn. The power source may be recharged using a variety of techniques depending on its configuration. Recharging the device can be accomplished via, without limitation, wireless induction, wireless resonance charging, a microbial solution, a chemical solution, etc.

With further reference to FIG. 1, the aligner 10 optionally includes one or more sensors 26. In an embodiment, the aligner 10 includes one or more temperature sensors that will automatically turn off the light source 20 if it senses the light source 20 is getting too hot (i.e., if it exceeds a predetermined temperature). In another embodiment, the aligner 10 includes one or more humidity sensors that will turn off the light source 20 and/or the power source 24 when the electronics come into contact with saliva in the mouth for example. In another embodiment, it may be desirable for the aligner 10 to include a sensor to detect initial placement of the aligner 10 on a patient's teeth. For example, such a sensor is useful in activating the light source 20 and/or the power source 24 when the aligner 10 is used for the first time. This prevents drainage of the power source 24 during a period of inactivity when the aligner 10 is not being used. Since multiple aligners representing various stages of treatment are not typically used concurrently, it will be useful to preserve the power source 24 of an aligner 10 prior to first use. In accordance with this embodiment, humidity sensors can be employed to turn on the light source 20 and/or the power source 24 when the electronics come into contact with the patient's mouth for example. In another embodiment, the aligner 10 includes a visible light sensor that causes the light source 20 and/or the power source 24 to automatically turn off when the device is outside of the mouth. The light source 20, power source 24, and any optional sensors 26 may be collectively referred to as the light therapy array 28. In another embodiment, the aligner 10 may include a manual on/off switch (not shown).

In an aspect of the present invention, any or all of the sensors (e.g., sensor 26) can be incorporated into a feedback system. The feedback loop may, for example, enable the user to know if optimum light emission is taking place and can measure the amount of time the light source 20 has been in use. Feedback can be delivered in any way, such as a website or a phone application, for the patient and/or orthodontist to access.

An embodiment of the present invention is directed to a method of fabricating a light assisted orthodontic device, such as the aligner 10. In one embodiment, the shell 12 may be made of an elastic material in one or more material layers. An exemplary shell 12 may include an inner material layer 30 and an outer material layer 32. Generally, the light therapy array 28 may be sealed within the aligner 10 by forming the inner material layer 30, coupling the light therapy array 28 to the inner material layer 30, and then forming the outer material layer 32 over the light therapy array 28 and the inner material layer 30. Accordingly, the light therapy array 28 may be embedded and permanently sealed within the aligner 10 such that the light therapy array 28 has no possibility of contact with bodily fluids and no possibility of being damaged by a patient accidentally biting down on the light therapy array or damaging the seal by other accidental means. It should be recognized that one or more components of the light therapy array 28 may optionally not be embedded in the aligner 10, as discussed above with regard to the power source 24. Further, embodiments of the present invention are not limited to shells 12 with two layers, as the shell 12 may include a single layer or more than two layers.

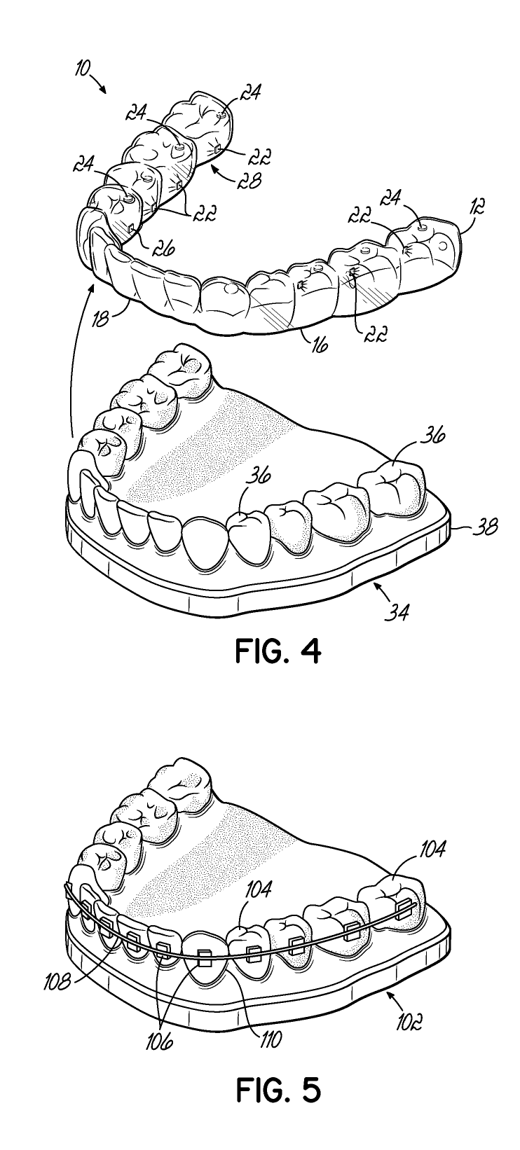

With reference to FIGS. 2A-2C, in one embodiment of the invention, a method for manufacturing the aligner 10 is shown. Generally, the aligner 10 includes two thermoplastic sheets that are used to form an inner material layer 30 and an outer material layer 32. In an embodiment, the thermoplastic sheets may have a thickness of 0.015 inch and may be made of, for example, polyurethane. As shown in FIG. 2A, a first thermoplastic sheet may be thermoformed over a positive mold 34 to form the inner material layer 30. The positive mold 34 includes a plurality of model teeth 36 arranged in accordance with a prescription on a base 38 that may include a model gingival margin 40. With reference to FIG. 2B, the light therapy array 28 is secured to the inner material layer 30. In an embodiment, the diodes 22 are connected via a flexible strip 41. A chemical bonding adhesive may be used to secure the light therapy array 28 on the inner material layer 30. This may be accomplished while the positive mold 34 remains in the thermoforming device (not shown). The light therapy array 28 may be embedded anywhere within the aligner 10. In an embodiment, the light therapy array 28 may be embedded on the lingual side of the inner material layer 30 (not shown), which may be advantageous cosmetically. However, for procedures where cosmetics are not an issue, such as night aligners, the light therapy array 28 may be located labially or in any location where the light source 20 may be more advantageously located. In the illustrated embodiment, the light therapy array 28 is embedded on the labial side 42 of the inner material layer 30. When the light therapy array 28 is securely in place, a second application of the chemical bonding adhesive may be used on the entire outer surface of the inner material layer 30. Next, as shown in FIG. 2C, a second thermoplastic sheet is thermoformed over the inner material layer 30 to form the outer material layer 32. Thus, as shown in FIG. 3, the light therapy array 28 is embedded securely and sealed within the aligner 10. With reference to FIG. 4, the aligner 10 may be separated from the positive mold 34 and may be prepared for further processing. For example, excess material at the gingival margin may be trimmed. A series of aligners may be made according to the same process. Each aligner is worn by the patient as determined by the dental practitioner, generally two weeks prior to being replaced by the next aligner in the sequence.

Other methods to secure and seal the light therapy array 28 within the aligner 10 or other light assisted orthodontic device are possible. For example, in an embodiment, a single 0.03 inch thick thermoplastic sheet may be fabricated with the light therapy array 28 positioned so that, when the thermoplastic sheet is thermoformed over the positive mold 34, the light therapy array 28 is located correctly in the single-layer aligner.

In another embodiment, the light therapy array 28 may be adhered to the lingual side or the labial side 42 of the aligner 10. The light therapy array 28 would be hermetically sealed in similarly transparent material against the surface of the aligner 10 to prevent damage or malfunction due to unplanned salivary exposure.

In an aspect of the present invention, the light assisted orthodontic device may be designed to protect the light therapy array from damage in use. For example, if the aligner 10 is a mandibular aligner and the light therapy array 28 is located on the lingual side, protecting the light therapy array 28 from teeth grinding is generally not required because it wouldn't generally come into contact with the opposing arch. However, if the aligner 10 is a maxillary aligner and the light therapy array 28 is on the lingual side, it may be constantly in contact with the mandibular anterior. For those patients who grind their teeth, the light therapy array 28 may require additional protection. Similarly, patients with severe malocclusions might be seen in digital treatment planning as a risk for damage to the light therapy array 28, and extra protection for the light therapy array 28 may be provided. In an embodiment, the protection may be in the form of a two-layer aligner having a thicker outer layer. In another embodiment, the protection may be in the form of an outer layer with differing thicknesses to provide a thicker side of the aligner expected to undergo more stress.

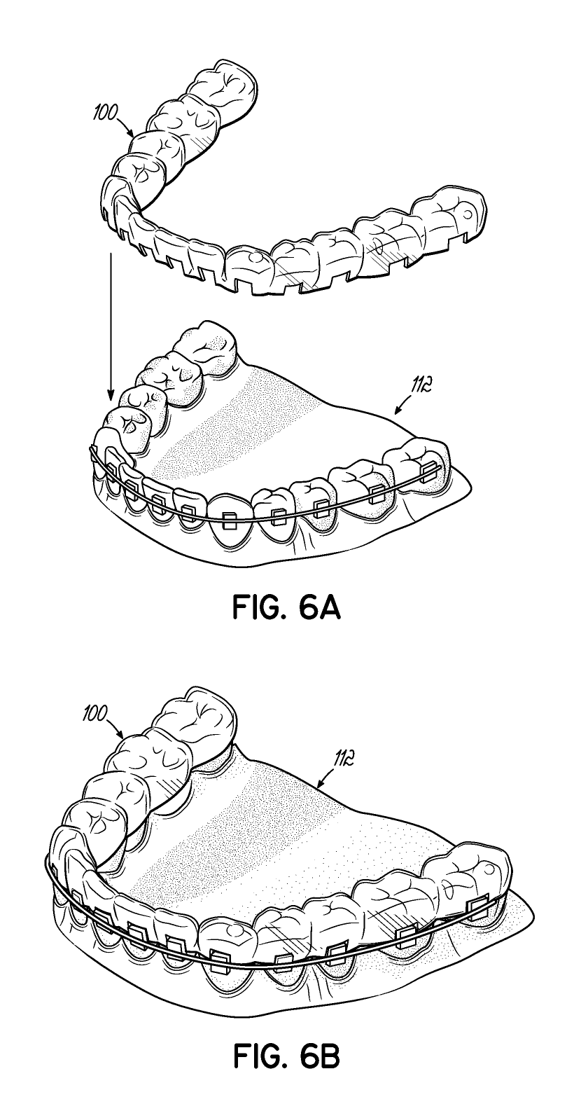

As described above, a light assisted orthodontic device may be used as an aligner during treatment. However, embodiments of the present invention are not so limited. For example, a light assisted orthodontic device may be worn in conjunction with traditional orthodontic appliances—i.e., brackets, archwires, etc. With reference to FIGS. 5-6B, in one embodiment, a light assisted orthodontic device is shown as a tray 100 configured to be worn over fixed orthodontic appliances (shown best in FIG. 5). Practical reasons may restrict the use of the device to nighttime use, although the treatment acceleration may be accomplished in the approximately eight hours that the tray 100 is worn nightly.

When the fixed orthodontic appliances are initially bonded to the teeth, the clinician may take an impression of the patient's teeth including the fixed appliances. By way of example only, and not limitation, an impression of the patent's teeth may be taken with a suitable dental impression material, such as polyvinylsiloxane (PVS). That dental impression may be scanned and the digital data from the scan may be imported into a computer to create a 3-D digital model of each of the patient's teeth. Alternatively, intra-oral images may be taken at the clinician's office using, for example, an intra oral scanner. Those images may then be used to produce a 3-D digital model of the patient's teeth. The 3-D digital model of the patient's teeth may be digitally manipulated to position each of the 3-D digital model teeth in a predetermined arrangement.

With reference to FIG. 5, a 3-D digital model 102 is shown. The digital model 102 includes digital teeth 104, brackets 106, and archwire 108. The digital model 102 may be cleaned of excess material using digital software tools available in software applications, such as Studio and Mudbox, so that each digital model 102 is a true representation of the patient's teeth and the fixed appliances. The 3-D digital model 102 may be used to design the light assisted orthodontic device (e.g., tray 100). Ease of placing and removing the tray 100 on the patient's teeth may be taken into account when designing the shape of the tray 100. For example, on the digital model 102, the entire labial and lingual areas from the archwire 108 to the gingival margin 110 may be virtually filled so that no undercuts remain.

Next, with reference to FIGS. 6A and 6B, the digital model 102 may be used to fabricate a physical model 112. In an embodiment, the digital model 102 is exported in STL format, and the physical model 112 is fabricated using a rapid prototyping device, such as a stereolithography machine. The tray 100 is then fabricated on the physical model 112. The tray 100 may be fabricated according to the methods described herein. Because the undercuts created by the archwire 108 were removed, the tray 100 will fit on the occlusal side of the patient's teeth and snuggly over the occlusal half of each bracket enabling the tray 100 to remain in place until removed.

In order to facilitate a more complete understanding of the embodiments of the invention, the following non-limiting example is provided.

### Example

An analytical method was devised to understand the penetration rate of oral tissues involved the oral tissue complex associated with orthodontic physiology—i.e., the gum (gingiva), cortical bone, and trabecular bone. The penetration rate is represented by the attenuation coefficient in the Beer-Lambert Law shown in Eq. 1. It is understood that penetration rate is inversely proportional to the attenuation coefficient.

$$T = \frac{{∅}_{T}}{{∅}_{i}} = {e}^{- α l} \qquad ( 1 )$$

where:

T is the penetration rate without the surface loss;

Ø_{T} is the radiant flux transmitted by the material, which represents the power measured on the back side of the tissue;

Øᵢ is the radiant flux received by the material;

α is the attenuation coefficient; and

l is the path length (or tissue thickness).

To establish a complete understanding of light transmission for photo-biomodulatory effects, it is assumed in the oral tissue complex that each tissue material (as represented in Beer-Lambert) has its own penetration rate.

Formulation of Prediction Equation.

Beer-Lambert's Law was modified to accommodate the assumption of surface loss of incident light at the initial tissue surface and the assumption that surface loss does not occur significantly at the inter-tissue surfaces, which resulted in the following equations relating the attenuation coefficient to the penetration rate:

$$\begin{matrix}T = \frac{{∅}_{T}}{{∅}_{0} ( 1 - r )} = {e}^{- α l} & ( 2 ) \\ {T}^{′} = \frac{{∅}_{T}}{{∅}_{0}} & ( 3 )\end{matrix}$$

where:

T, Ø_{T}, α, and l are described above for Eq. 1;

T′ is the measured penetration rate;

Ø₀ is the incoming radiant flux, which represents the power measured without the sample tissue; and

r is the surface loss rate.

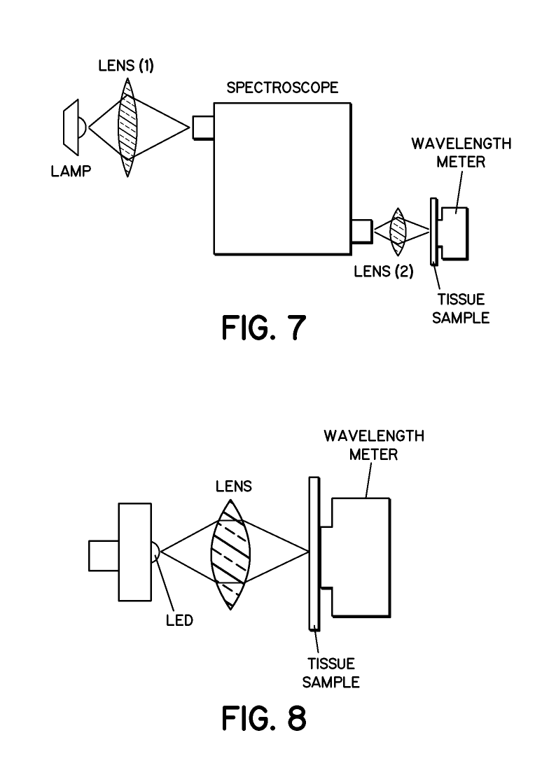

Tissue samples from Yucatan pigs were used to represent human oral tissues. Samples were prepared in 1 mm thick biological sections. These tissue samples were tested across a wavelength spectrum of 420 nm to 700 nm emitted from 100 W LED white light source. These tissues were also tested at higher wavelengths, 700 nm to 1050 nm, using infrared LED light sources. FIGS. 7 and 8 show the test setup. A spectroscope was used to specify the input wavelength to be tested.

Two pieces of tissue samples of the same material from the same animal were prepared. The thickness, l₁ and l₂, were measured. The input light power (Ø₀) was measured using the wavelength meter as shown in FIGS. 7 and 8. The first piece of tissue sample was attached at the entrance of the wavelength meter for the test. The optical power transmitted through the material was measured (Ø_{1T}). Next, the second piece of tissue sample was attached to the back surface of the first piece. Then the optical power transmitted by the two pieces of material was measured (Ø_{2T}). The attenuation coefficient (α) and surface loss rate (r) of this material can be obtained by simultaneously solving Eqs. 4 and 5, which result in Eqs. 6 and 7:

$$\begin{matrix}\frac{{∅}_{1 T}}{{∅}_{0} ( 1 - {r}_{1} )} = {e}^{- α {l}_{1}} & ( 4 ) \\ \frac{{∅}_{2 T}}{{∅}_{0} ( 1 - {r}_{1} )} = {e}^{- α ( {l}_{1} + {l}_{2} )} & ( 5 ) \\ α = \frac{1}{{l}_{2}} ln ( \frac{{∅}_{1 T}}{{∅}_{2 T}} ) & ( 6 ) \\ {r}_{1} = 1 - \frac{{∅}_{1 T}}{{∅}_{0} {e}^{- α {l}_{1}}} & ( 7 )\end{matrix}$$

Validation of the Prediction Equation.

To determine the effects of light power on the penetration rate, a test was performed to validate the hypothesis that the attenuation coefficient or penetration rate is not affected by the strength of the incoming light power. Light with different intensities (power/unit area) was applied to the same sample. The optical intensity was adjusted from low to high within the range of 0.3 to 8.8 mW/cm² and the penetration rates were recorded and compared.

Once the penetration rate of each individual tissue was determined, the penetration rate of combined tissues was estimated using the Beer-Lambert law. A validation test was performed to prove the hypothesis that the penetration rate of tissue combination is equal to the product of the penetration rate of each individual rate with the surface loss effect taken into consideration. The hypothesis can be represented by the following equation:

$${T}_{c}^{′} = \frac{{∅}_{2 T}}{{∅}_{o}} = ( 1 - {r}_{1} ) {e}^{- {α}_{1} {l}_{1}} {e}^{- {α}_{2} {l}_{2}} \qquad ( 8 )$$

where:

T_{c} is the penetration rate of the combined tissues;

r₁ is the surface loss rate of individual tissue experiencing incident light;

α₁ and α₂ are the attenuation coefficients of corresponding tissues; and

l₁ and l₂ are the thicknesses of corresponding tissues.

To validate the hypothesis, the penetration rates of two tissue combinations-1) gum and cortical bone and 2) cortical bone and trabecular bone—were measured and then compared with the predicted penetration rates that were calculated based on the penetration rates of the corresponding single materials from the same animal using Eq. 7. The experiment consisted of stacking the two flat pieces of different tissue samples and measuring the penetration rate.

To further validate the hypothesis, the penetration rate of combined tissues (tissue complex) consisting of the gum, cortical bone, and trabecular bone in natural anatomy harvested pigs were tested and compared with the prediction. The thickness of each tissue in pig sample was measured using a caliper at the nearby sections.

Results.

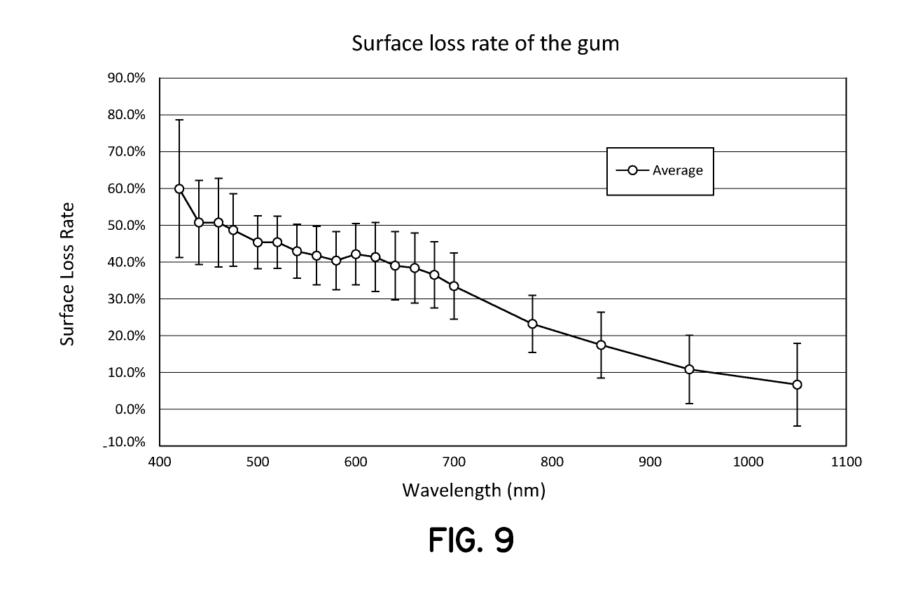

The surface loss results are shown in FIG. 9. A measurable fraction of the incident light was lost at the gum surface. Loss fraction as a function of wavelength graphically demonstrates much greater loss at lower wavelengths, lesser at the higher end as the wavelength approached 1050 nm.

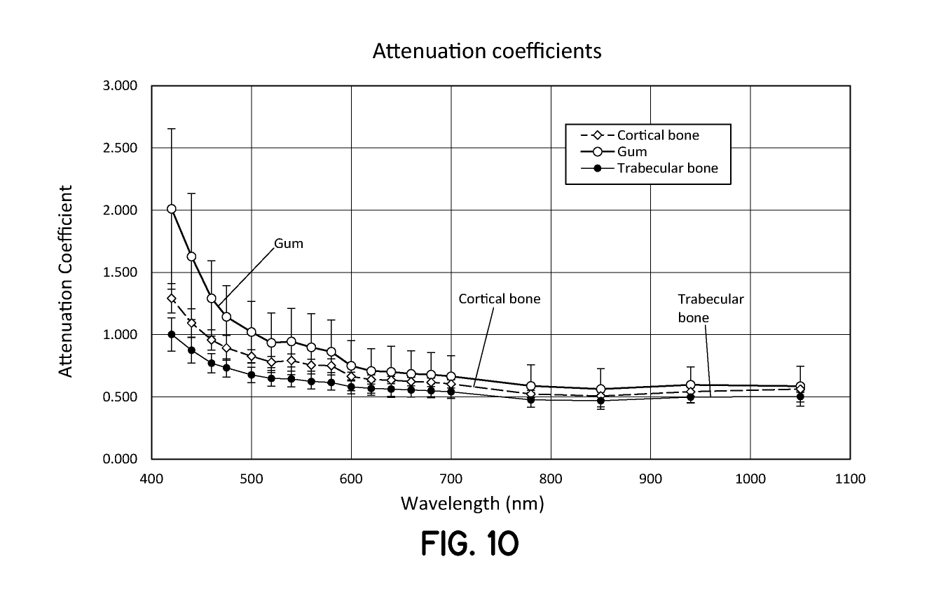

The measured attenuation coefficients are shown in FIG. 10. The results demonstrated that the penetration rate (as defined by attenuation coefficient) depends at least in part on wavelength. As the wavelength increases over the analytical range, the attenuation coefficient falls significantly to about 0.5 for all tissues. In that regard, the penetration rates optimize and plateau at wavelengths exceeding 700 nm. It was also determined that gum tissue had nearly double (about 2 to 1) the attenuation coefficient of trabecular bone at the lowest wavelengths. The attenuation coefficient of the cortical bone was measured near 1.3. Deviations outside of the range of 700 nm to 1500 nm lead to inefficiencies such as reflection, scattering, or premature absorbance.

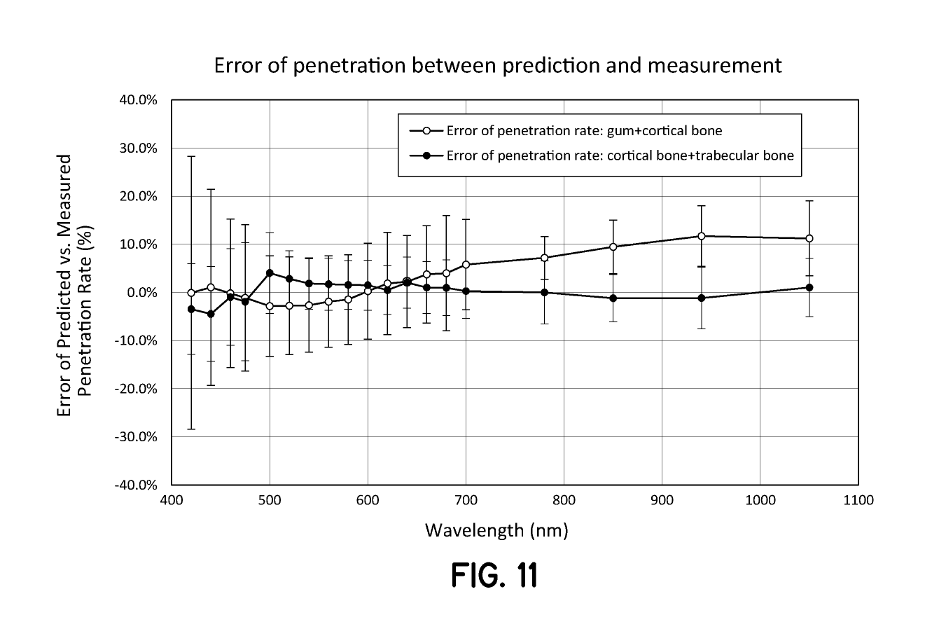

Next, the derived Beer-Lambert law for stacked tissues (Eq. 7) was applied to estimate the penetration rate of two different tissues in the complex. A plot of error as a function of wavelength for experimental versus prediction demonstrates validation of the law by the proposed analytical method is shown in FIG. 11. Two different stack combinations were plotted: 1) gum and cortical bone and 2) cortical and trabecular bones, the latter having a lower error. Both error plots remained within 10% for nearly the entire range.

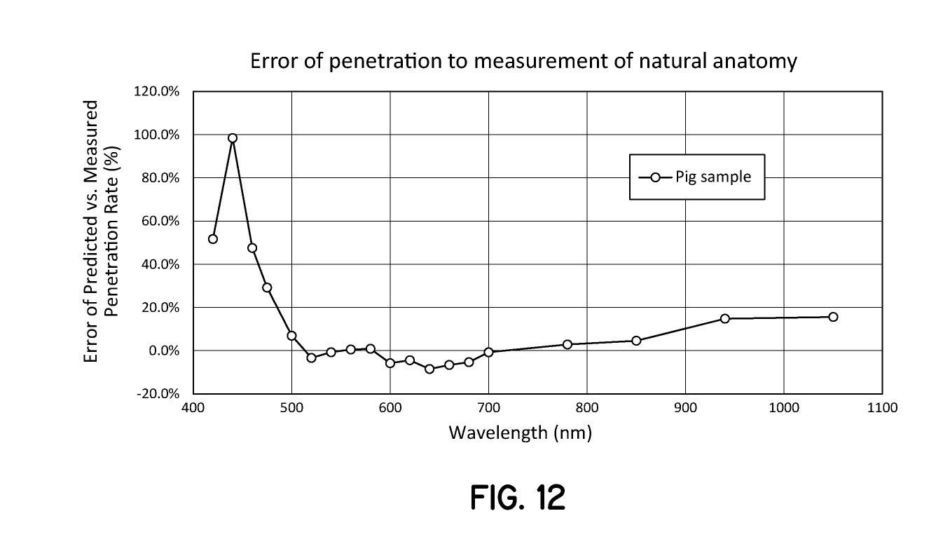

A final validation step was performed on the complete tissue complex within the natural anatomy of a pig, and the results are shown in FIG. 12. The prediction was validated for all wavelengths with exception to less than 500 nm. Wavelengths in the near-infrared (NIR) region (e.g., 780 nm to 2500 mm) were especially predictive. The error may be accounted for in the random selection of tissues from 1 of 10 pigs, where an average attenuation coefficient for all 10 was used for prediction. Further, the attenuation coefficients at the lower wavelengths (e.g., less than 500 nm) were highly variable among the individual tissues. Thus, the lower wavelength predictions in the complex were highly sensitive to tissue thickness, which is difficult to control.

Finally, the method was used to measure penetration rate as a function of light intensity. Three pieces of cortical bone, one trabecular bone, and one gum were tested at two distinct wavelengths and wide-ranging intensities (i.e., 0.3 mW/cm² to 8.8 mW/cm², or 300 μW/cm² to 8,800 μW/cm²). The results are shown in Tables 1 and 2 below. It was experimentally determined that penetration rate changes are negligible (i.e., less than 3.5%) with intensity.

TABLE 1

| Power | | Power | | Power | |
|---|---|---|---|---|---|
| (μW/cm²) at | Cortical | (μW/cm²) | Cortical | (μW/cm²) | Cortical |
| 940 nm | 1 | at 940 nm | 2 | at 780 nm | 3 |
| 310 | 32.0% | 586 | 32.3% | 1281 | 29.4% |
| 840 | 30.9% | 854 | 33.8% | 1770 | 30.3% |
| 1206 | 29.5% | 1178 | 33.7% | 2432 | 31.1% |
| 1881 | 31.5% | 1633 | 33.1% | 3100 | 30.7% |
| 1950 | 31.2% | 2184 | 32.7% | 3699 | 30.5% |
| 2824 | 33.1% | 3038 | 32.1% | 4788 | 30.7% |
| 3575 | 30.7% | 3782 | 33.5% | 6048 | 30.4% |
| 4795 | 30.9% | 6131 | 32.9% | — | — |
| 5298 | 30.9% | — | — | — | — |
| 6131 | 30.9% | — | — | — | — |
| Correlation | −0.091 | Correlation | −0.127 | Correlation | 0.353 |

TABLE 2

| Power | | Power | |
|---|---|---|---|
| (μW/cm²) at | | (μW/cm²) at | |
| 940 nm | Gum | 780 nm | Trabecular |
| 413 | 29.3% | 1005 | 18.0% |
| 923 | 28.7% | 1642 | 17.8% |
| 1467 | 29.1% | 2455 | 17.9% |
| 2129 | 28.7% | 3589 | 18.4% |
| 2563 | 29.1% | 5027 | 17.8% |
| 3376 | 29.2% | 6048 | 17.8% |
| 6510 | 28.6% | 7256 | 18.2% |
| 8797 | 29.1% | — | — |
| Correlation | −0.212 | Correlation | 0.147 |

As shown in the results, the Beer-Lambert law with incident surface loss effect taken into consideration (Eq. 7) is a strong basis to estimate light penetration rate of complexes including multiple types of oral tissue. The derived Beer-Lambert law was built using the assumptions that penetration rates are significantly influenced by the surface loss at the incident light interface and are insignificantly influenced at the tissue interfaces. In that regard, the penetration rates of two stacked tissues and of the entire tissue complex were predicted with low error using the derived Beer-Lambert law. Additionally, the results demonstrated that penetration rates increase significantly at longer wavelengths. For example, penetration rates of gum tissue increased from 13.4% to 55.7% (+4χ) with a wavelength of 420 nm compared to 1050 nm. Penetration rates of these oral tissues converge and plateau between 700 and 1050 nm. Thus, wavelengths of 700 nm to 1050 nm, which includes NIR, may be an optimum spectral range for photo-biomodulation based upon this method. Further, penetration rates were not significantly influenced by light intensity as demonstrated in the range of 0.3 mW/cm² to 8.8 mW/cm². Thus, a device emitting light at one wavelength may be equally effective at relatively low power compared to high power. The Beer-Lambert law with incident surface loss effect taken into consideration is a strong basis to estimate light penetration rate of a complex including multiple types of oral tissue.

In addition to the tests described above with pig samples, human samples were also tested. The results generally confirmed those of the pig sample tests. A portion of the light intensity was lost at the surface of the gum. The human sample demonstrated a greater loss at lower wavelengths, which generally plateaued at wavelengths exceeding 700 nm and slowly lessened as the wavelength approached 1050 nm. In that regard, the results demonstrated that penetration rates increase significantly at longer wavelengths. The penetration rates of two stacked tissues and of the entire tissue complex were predicted with low error using the derived Beer-Lambert law. Compared to pig's tissue, the human samples showed higher attenuation coefficient, especially for trabecular bone, although this may have been due to differences in sample preparation.

While the present invention has been illustrated by a description of various embodiments and while these embodiments have been described in some detail, it is not the intention of the inventors to restrict or in any way limit the scope of the appended claims to such detail. Thus, additional advantages and modifications will readily appear to those of ordinary skill in the art. The various features of the invention may be used alone or in any combination depending on the needs and preferences of the user.

What is claimed is:

1. A light assisted orthodontic device comprising:
at least one material layer formed to be positioned over one or more teeth;
a light source coupled to the at least one material layer, the light source being configured to emit light having a wavelength of 700 nm to 1500 nm; and
a power source configured to power the light source.

2. The device of claim 1, wherein the light source is configured to emit light having a wavelength of 700 nm to 1050 nm.

3. The device of claim 1, wherein the light source is configured to emit light having a dose in a range from 1 J/cm² to less than 5 J/cm².

4. The device of claim 1, wherein the light source is configured to emit light having an intensity in a range from 0.3 mW/cm² to 8.8 mW/cm².

5. The device of claim 1, wherein the light source is coupled to a flat or flexible substrate.

6. The device of claim 1, wherein the light source is configured to emit light having a greater wavelength in a posterior region of the device compared to an anterior region of the device.

7. The device of claim 1, further comprising:
a sensor to detect initial placement of the device on a patient's teeth.

8. The device of claim 7, wherein the sensor is configured to activate the light source when the device is placed on the patient's teeth.

9. The device of claim 1, wherein the light source is coupled to a first material layer and a second material layer covers the light source and the first material layer.

10. The device of claim 1, further comprising:
one or more temperature sensors configured to automatically turn off the light source if it exceeds a predetermined temperature.

11. The device of claim 1, wherein the power source is a micro battery.

12. The device of claim 11, wherein the micro battery includes at least one carbon fluoride cathode and at least one lithium anode.

13. The device of claim 1, wherein the device is configured to be positioned over one or more fixed orthodontic appliances.

14. A method for making a light assisted orthodontic device comprising:
forming a first material layer shaped to be positioned over one or more teeth; and
coupling a light therapy array to the first material layer, the light therapy array comprising a light source configured to emit light having a wavelength of 700 nm to 1500 nm.

15. The method of claim 14, wherein the light source is configured to emit light having a wavelength of 700 nm to 1050 nm.

16. The method of claim 14, further comprising:
forming a second material layer over the first material layer and light therapy array.

17. The method of claim 14, wherein the second material layer is thicker than the first material layer.

18. The method of claim 14, wherein the light therapy array is coupled to the first material layer before the first material layer is shaped, the light therapy array being positioned so that, when the thermoplastic sheet is shaped, the light therapy array is located in a predetermined position in the shaped first material layer.

19. The method of claim 14, further comprising:
printing the light source on a flat or flexible substrate via thin-film processing.

20. A method of orthodontic treatment using a light assisted orthodontic device comprising:
positioning the device over one or more teeth, the device applying a corrective pressure to the one or more teeth; and
emitting light having a wavelength of 700 nm to 1500 nm to the one or more teeth.

21. The method of claim 20, wherein the light has a dose in a range from 1 J/cm² to less than 5 J/cm².

22. The method of claim 20, wherein the light has an intensity in a range from 0.3 mW/cm² to 8.8 mW/cm².

23. The method of claim 20, wherein emitting light includes emitting light having a greater wavelength in a posterior region of the device compared to an anterior region of the device.

24. The method of claim 20, wherein the device includes a sensor, the method further comprising:
detecting initial placement of the device on a patient's teeth using the sensor; and
activating the light source when the device is placed on the patient's teeth.

25. The method of claim 20, further comprising:
automatically turning off the light source if it exceeds a predetermined temperature.

26. A method for determining a penetration rate of light through oral tissue comprising:
providing a light source;
directing light from the light source onto a sample of oral tissue;
measuring a first power of the light at a first surface of the sample;
measuring a second power of the light at a second surface of the sample;
computing a surface loss of the light; and
determining the penetration rate of the light using the first power, the second power, and the surface loss.
